(19) United States
(12) Patent Application Publication
(10) Pub. No.: US 2010/0076047 A1
(43) Pub. Date: Mar. 25, 2010

(54) Amorphous 1-(9H-carbazol-4-yloxy)-3-[[2-(2-methoxyphenoxy)ethyl]amino]-2-propanol phosphate salt

(76) Inventors: Sankar Reddy Budidet, Hyderabad (IN); Yallappa Somappa Somannavar, Hyderabad (IN); Ramesh Dandala, Hyderabad (IN); Sivakumaran Meenakshisunderam, Hyderabad (IN)

Correspondence Address: Jay R Akhave, #612, 2058 N Mills Aave, Clarement, CA, 91711, US

(21) Appl. No.: 12/229,170

(22) Filed: Aug. 20, 2008

(30) Foreign Application Priority Data
Aug. 20, 2007 (IN) 1844/CHE/2007

(51) Int. Cl. C07D 209/82; A61K 31/403; A61P 9/12

(52) U.S. Cl. 514/411; 548/444

(57) ABSTRACT

The present invention relates to a novel amorphous form of 1-(9H-carbazol-4-yloxy)-3-[[2-(2-methoxyphenoxy)ethyl]amino]-2-propanol phosphate (Carvedilol dihydrogen phosphate) of Formula (I), and a process for the preparation thereof.

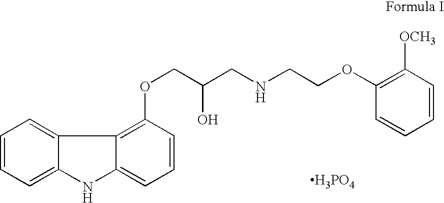

### CROSS REFERENCE TO THE RELATED APPLICATION

This application claims the priority of an Indian Patent application No. 1844/CHE/2007 filed on Aug. 20, 2007.

### FIELD OF THE INVENTION

The present invention relates to a novel amorphous form of 1-(9H-carbazol-4-yloxy)-3-[[2-(2-methoxyphenoxy)ethyl]amino]-2-propanol phosphate (Carvedilol dihydrogen phosphate) of Formula (I), and a process for the preparation thereof.

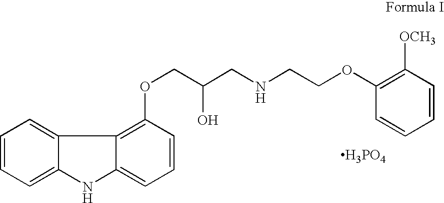

### BACKGROUND OF THE INVENTION

1-(9H-Carbazol-4-yloxy)-3-[[2-(2-methoxyphenoxy)ethyl]amino]-2-propanol (II) is generically known as Carvedilol. Carvedilol and its pharmaceutically acceptable salts are nonselective β-adrenergic blocker with α₁-blocking activity. Carvedilol is used for the treatment of hypertension, congestive heart failure and angina.

Carvedilol is approved as conventional tablets as well as controlled release tablets. The conventional tablet contains Carvedilol as the active ingredient and is administered twice daily. The recently approved controlled release dosage form contains Carvedilol dihydrogen phosphate as the active ingredient and is administered once a day.

Boehringer Mannheim, GmbH, has disclosed Carvedilol and its pharmaceutically acceptable salts in U.S. Pat. No. 4,503,067. The aforementioned free base form of Carvedilol is a racemic mixture of R(+) and S(−) enantiomers, where nonselective β-adrenoreceptor blocking activity is exhibited by the S(−) enantiomer and α-adrenergic blocking activity is exhibited by both R(+) and S(−) enantiomers. Those unique features or characteristics associated with such a racemic Carvedilol mixture contributes to two complementary pharmacologic actions: i.e., mixed venous and arterial vasodilation and non-cardioselective, β-adrenergic blockade

Different forms of a pharmaceutically useful compound provide opportunities to improve the performance characteristics of a pharmaceutical product. Different forms enlarge the repertoire of materials that a formulation scientist has available for designing, for example, a pharmaceutical dosage form of an active pharmaceutical ingredient with a desired characteristic. It is well known that new forms of known useful compounds are of utility. Consequently, there is an ongoing search for new forms of known compounds used in pharmaceutical compositions, which may provide for improved performance thereof.

US 2005-0240027 A1 by Brook et al., (Smithkline Beecham) discloses the Carvedilol phosphate salts, which include crystalline forms of Carvedilol phosphate such as Carvedilol dihydrogen phosphate hemihydrate, dihydrate, Carvedilol hydrogen phosphate and other corresponding solvates thereof such as methanol solvate. This patent publication also discloses compositions containing such salts and/or solvates, and methods of using the aforementioned compounds to treat hypertension, congestive heart failure and angina, etc.

It has been disclosed that amorphous forms of a number of drugs exhibit different dissolution characteristics and in some cases different bioavailability patterns compared to the corresponding crystalline forms [Konne T., Chem. Pharm. Bull., 38, 2003 (1990)]. For some therapeutic indications one bioavailability pattern may be favoured over another.

Therefore, there is a need for the development of an amorphous form of Carvedilol phosphate and improved process for the preparation thereof.

Amorphous Carvedilol dihydrogen phosphate is stable when stored under controlled humidity conditions and can be formulated into a suitable dosage form without conversion to a crystalline form. Solid amorphous Carvedilol dihydrogen phosphate is provided herein. The amorphous Carvedilol dihydrogen phosphate can be produced with no detectable crystalline Carvedilol dihydrogen phosphate present based on XRD investigations and has a superior stability profile compared to the existing crystalline forms.

### OBJECTIVE OF THE INVENTION

The main objective of the present invention is to provide stable amorphous form of Carvedilol phosphate and a process for the preparation thereof in high purity and yield on a commercial scale.

### SUMMARY OF THE INVENTION

The present invention relates to an amorphous form of 1-(9H-carbazol-4-yloxy)-3-[[2-(2-methoxyphenoxy)ethyl]amino]-2-propanol phosphate salt (Carvedilol dihydrogen phosphate) Formula (I).

The novel amorphous form of Carvedilol dihydrogen phosphate can be characterized by XRD data as shown in FIG. 1.

Another embodiment of the present invention further relates to the process for the preparation of amorphous form of 1-(9H-carbazol-4-yloxy)-3-[[2-(2-methoxyphenoxy)ethyl]amino]-2-propanol phosphate salt (Carvedilol dihydrogen phosphate) Formula (I),

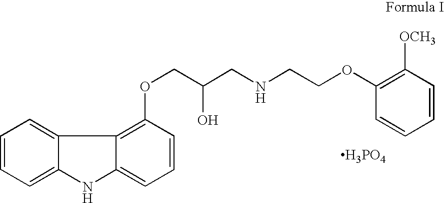

which comprises,
i) reacting 1-(9H-carbazol-4-yloxy)-3-[[2-(2-methoxyphenoxy)ethyl]amino]-2-propanol (II)

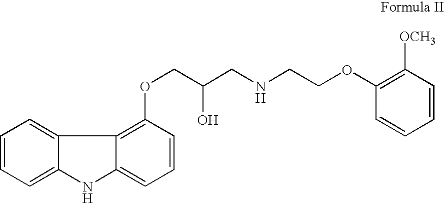

with phosphoric acid of formula (III).

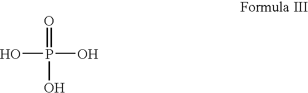

ii) isolating amorphous Carvedilol dihydrogen phosphate

In another embodiment, the present invention provides a pharmaceutical composition comprising a therapeutically effective amount of the novel amorphous form of Carvedilol dihydrogen phosphate, with pharmaceutically acceptable carriers.

### BRIEF DESCRIPTION OF THE DRAWINGS

FIG. 1 is an X-ray powder diffraction pattern of novel amorphous form of Carvedilol dihydrogen phosphate.

### DETAILED DESCRIPTION OF THE INVENTION

The present invention relates to an amorphous form of 1-(9H-carbazol-4-yloxy)-3-[[2-(2-methoxyphenoxy)ethyl]amino]-2-propanol phosphate salt (Carvedilol dihydrogen phosphate) Formula (I).

The present invention further relates to a process for the preparation of amorphous form of 1-(9H-carbazol-4-yloxy)-3-[[2-(2-methoxyphenoxy)ethyl]amino]-2-propanol phosphate salt (Carvedilol dihydrogen phosphate) of Formula (I), by reacting Carvedilol with orthophosphoric acid. in a solvent at a temperature of about 20-50° C.

The process comprises dissolving orthophosphoric acid in a solvent and removing water. The solvent is selected from ethers such as THF, diethylether, dimethyl ether, methylethyl ether; esters such as ethyl acetate, n-propyl acetate, isopropyl acetate; aromatic hydrocarbons such as benzene, toluene, xylene; nitriles such as acetonitrile, propyl nitrile, butyl nitrile; chlorinated hydrocarbons such as methylene chloride, ethylene dichloride, chloroform; ketones such as acetone, methyl ethyl ketone, 2-pentanone; alcohols such as methanol, ethanol, isopropanol, n-propanol, n-butanol, t-butanol and mixtures thereof, which are capable of forming azeotropic mixture with water to remove water present in the orthophosphoric acid. Alternatively anhydrous orthophosphoric acid can be used directly. The above resulting phosphoric acid residue is treated with Carvedilol in a organic solvent selected from ethers such as THF, diethyl ether, dimethyl ether, methylethyl ether; esters such as ethyl acetate, n-propyl acetate, iso-propyl acetate; aromatic hydrocarbons such as benzene, toluene, xylene, heptane; nitriles such as acetonitrile, propyl nitrile, butyl nitrile; chlorinated hydrocarbons such as methylene chloride, ethylene dichloride, chloroform; ketones such as acetone, methyl ethyl ketone, 2-pentanone; alcohols such as methanol, ethanol, isopropanol, n-propanol, n-butanol, t-butanol and mixtures thereof at a temperature of about room temperature to reflux temperature, to produce amorphous Carvedilol dihydrogen phosphate. The amorphous Carvedilol dihydrogen phosphate is isolated from the reaction mixture by techniques which may be employed to isolate amorphous Carvedilol dihydrogen phosphate from the solution include those wherein the precipitate may be separated by techniques well known in the art. Preferably, the precipitate is separated by filtration. Optionally, vacuum filtration may be utilized.

Techniques which may also be employed to isolate amorphous Carvedilol dihydrogen phosphate from the solution include those wherein the solvent is removed from the solution, preferably rapidly, and leave the product deposited. Methods involving the use of these procedures, which have been found to be satisfactory, include spray drying, roller drying, rotary evaporation, and freeze-drying.

A preferred method of removing the solvent, suitable for preparing amorphous Carvedilol dihydrogen phosphate from the solution is by spray drying. In spray drying, a solution is sprayed from a nozzle into or with a large volume of a gas such as air. Generally, the solution, the nozzle and/or the gas are heated. The spraying from the nozzle generates droplets of solution containing substances in the solution. The combination of gas and heat causes the solvents in the droplets to evaporate, leaving an amorphous form of the substances to be gathered.

The amorphous product produced by the present invention is stable when stored under controlled humidity conditions and can be formulated into a suitable dosage form without conversion to a crystalline form. The amorphous Carvedilol dihydrogen phosphate contains up to 7% moisture and can be hemihydrate, monohydrate or dihydrate. The amorphous Carvedilol dihydrogen phosphate of low moisture content when exposed to air picking-up moisture up to 7% forming higher hydrates. Even then, the XRD pattern remains identical.

The present invention also relates to a pharmaceutical composition comprising an effective amount of amorphous Carvedilol dihydrogen phosphate, in association with one or more non-toxic pharmaceutically acceptable carriers and/or diluents thereof, and if desired, other active ingredients and the quantity of the compound or composition of the present invention administered will vary depending on the patient and the mode of administration and can be any effective amount.

In particular, a composition of the present invention is presented as a unit dose and taken preferably from 1 to 2 times daily, most preferably once daily to achieve the desired effect.

In general, pharmaceutical compositions of the present invention are prepared using conventional materials and techniques, such as mixing, blending and the like.

According to the present invention, pharmaceutical composition containing amorphous Carvedilol dihydrogen phosphate can also include, but are not limited to, suitable adjuvants, carriers, excipients, or stabilizers, etc. and can be in solid or liquid form such as, tablets.

The present invention also relates to method of treating hypertension, congestive heart failure, angina, comprising administering to a mammal in need thereof therapeutically effective amount of amorphous Carvedilol dihydrogen phosphate.

The details of the process of the invention are provided in the examples given below, which are provided by way of illustration only and therefore should not be construed to limit the scope of the invention.

### EXAMPLE-1

Preparation of amorphous form of 1-(9H-carbazol-4-yloxy)-3-[[2-(2-methoxyphenoxy)ethyl]amino]-2-propanol phosphate salt (Carvedilol dihydrogen phosphate).

Orthophosphoric acid (6.02 g, 88%, 0.0541 mol) was added to toluene (200 ml) and distilled out water azeotropically under stirring at a temperature of about 95-110° C. and then cooled to 30-40° C. to give orthophosphoric acid residue. Methylene chloride (100 ml) was added to the residue under nitrogen atmosphere and heated to 38-42° C. A solution of Carvedilol (20 g, 0.0492 mol) in methylene chloride (200 ml) prepared at a temperature of about 38-42° C., was added to the orthophosphoric acid residue at 38-42° C. and stirred the reaction mass for 8 h at a temperature of about 38-42° C. The resulting solid product was filtered under nitrogen atmosphere and dried at a temperature of about 40-50° C. to yield (22 g) amorphous Carvedilol dihydrogen phosphate.

### EXAMPLE-2

Preparation of amorphous form of 1-(9H-carbazol-4-yloxy)-3-[[2-(2-methoxyphenoxy)ethyl]amino]-2-propanol phosphate salt (Carvedilol dihydrogen phosphate).

Crystalline phosphoric acid (26.51 g, 0.2705 mol) was added to toluene (600 ml) and distilled out toluene under stirring at reflux temperature and then cooled to 30-40° C. to give orthophosphoric acid residue. Methylene chloride (300 ml) was added to the residue under nitrogen atmosphere and heated to a temperature of about 38-42° C. A solution of Carvedilol (100 g, 0.246 mol) in methylene chloride (1000 ml) prepared at a temperature of about 38-42° C., was added to the orthophosphoric acid residue in methylene chloride at a temperature of about 38-42° C. and stirred the reaction mass for 6 h at a temperature of about 38-42° C. The resulting solid product was filtered under nitrogen atmosphere and dried at a temperature of about 40-50° C. to yield (109 g) amorphous Carvedilol dihydrogen phosphate.

We claim:

1) Amorphous 1-(9H-carbazol-4-yloxy)-3-[[2-(2-methoxyphenoxy)ethyl]amino]-2-propanol phosphate.

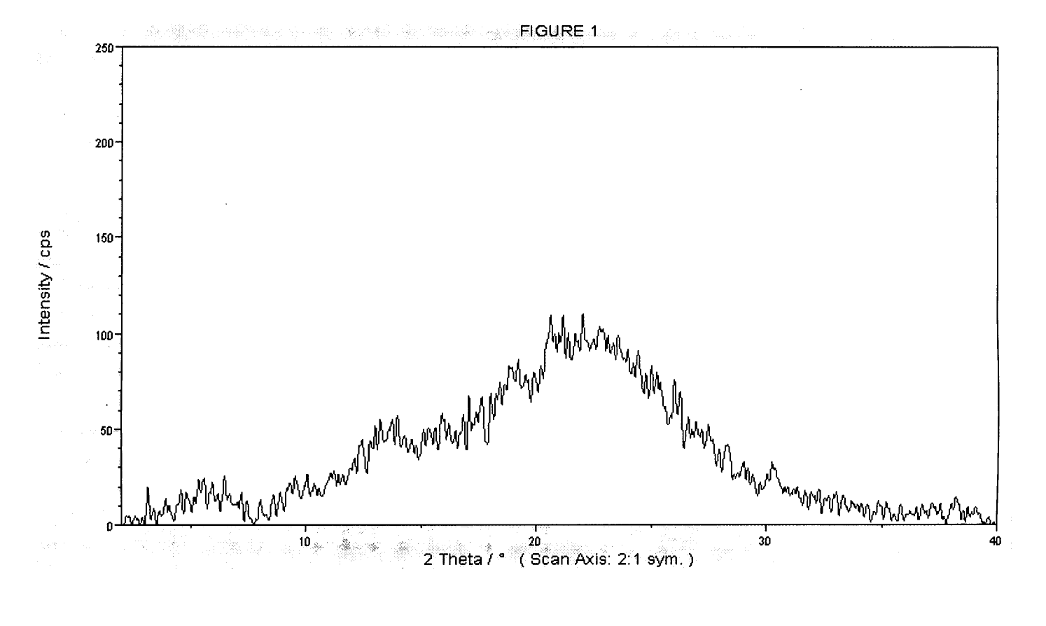

2) Amorphous Carvedilol dihydrogen phosphate as claimed in claim 1 having the X-ray diffraction spectrum as depicted in FIG. 1.

3) A process for the preparation of amorphous form of 1-(9H-carbazol-4-yloxy)-3-[[2-(2-methoxyphenoxy)ethyl]amino]-2-propanol phosphate salt (Carvedilol dihydrogen phosphate) Formula (I), 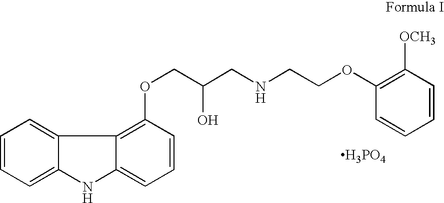
which comprises,
i) reacting 1-(9H-carbazol-4-yloxy)-3-[[2-(2-methoxyphenoxy)ethyl]amino]-2-propanol (II) 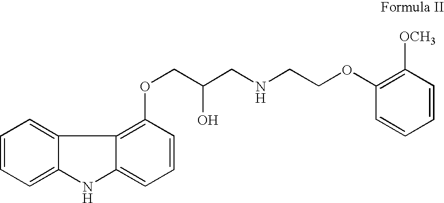
with phosphoric acid of formula (III) 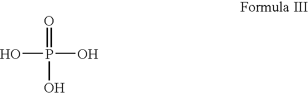
ii) isolating amorphous Carvedilol dihydrogen phosphate.

4) The process of claim 3, wherein the reaction comprises;
i) treating the orthophosphoric acid with a solvent;
ii) removing the water from orthophosphoric acid to produce anhydrous phosphoric acid residue;
iii) treating the phosphoric residue with Carvedilol in a solvent to produce amorphous form of Carvedilol dihydrogen phosphate;
iv) isolating the amorphous Carvedilol hydrogen phosphate.

5) The process of claim 4 wherein the solvent used in step (i) & (iii) is selected from ethers, esters, aromatic hydrocarbons, nitriles, chlorinated hydrocarbons, ketones, alcohols and mixtures thereof.

6) The process of claim 5 wherein the ethers are selected THF, diethylether, dimethyl ether, methylethyl ether, methylpropyl ether, methylbutyl ether, MTBE, or mixtures thereof.

7) The process of claim 5 wherein the esters are selected from ethylacetate, propyl acetate, phenylacetate or mixtures thereof.

8) The process of claim 5 wherein the aromatic hydrocarbons are selected from benzene, toluene, xylene, heptane, hexanes or mixtures thereof.

9) The process of claim 5 wherein the nitriles are selected from acetonitrile, propyl nitrile, butyl nitrile, pentane nitrile, benzonitrile or mixtures thereof.

10) The process of claim 5 wherein the chlorinated hydrocarbons are selected from methylene chloride, diethylene chloride, chloroform or mixtures thereof.

11) The process of claim 5 wherein the ketones are selected from acetone, methyl ethyl ketone, 2-pentanone, 3-pentanone, acetophenone or mixtures thereof.

12) The process of claim 5 wherein the alcohols are selected from methanol, ethanol, isopropanol, n-propanol, n-butanol, t-butanol or mixtures thereof.

13) The process of claim 4, wherein the isolation of amorphous Carvedilol dihydrogen phosphate from the solution includes by the separation techniques such as filtration.

14) The process of claim 4, wherein the isolation of amorphous Carvedilol dihydrogen phosphate from the solution includes removing the solvent from the solution, by the techniques such as spray drying, roller drying, rotary evaporation, and freeze-drying.

15) The process of claim 4, wherein the amorphous Carvedilol dihydrogen phosphate is isolated from the solution by spray drying technique.

16) A pharmaceutical composition comprising amorphous Carvedilol dihydrogen phosphate of claim 1 and a pharmaceutically acceptable carrier and/or diluents.

17) A method of treating hypertension, congestive heart failure and angina, comprising administering a therapeutically effective amount of amorphous Carvedilol hydrogen phosphate of claim 1.
